(19) United States
(12) Patent Application Publication
(10) Pub. No.: US 2010/0113517 A1
(43) Pub. Date: May 6, 2010

(54) METHOD FOR THE TREATMENT OF FABRY DISEASE USING PHARMACOLOGICAL CHAPERONES

(75) Inventor: David Palling, Upper Montclair, NJ (US)

Correspondence Address: HOFFMANN & BARON, LLP, 6900 JERICHO TURNPIKE, SYOSSET, NY, 11791, US

(73) Assignee: AMICUS THERAPEUTICS, INC., Cranbury, NJ (US)

(21) Appl. No.: 12/594,124

(22) PCT Filed: Mar. 28, 2008
(86) PCT No.: PCT/US08/58668

§ 371 Date: Sep. 30, 2009

Related U.S. Application Data
(60) Provisional application No. 60/909,185, filed on Mar. 30, 2007.

(51) Int. Cl. A61K 31/445; A61P 9/10

(52) U.S. Cl. 514/315

(57) ABSTRACT

The present invention provides a method treating a patient with Fabry disease by determining whether there is an improvement of a surrogate marker that is associated with Fabry disease following administration of a specific pharmacological chaperone of α-galactosidase A. The method includes administering an effective amount of 1-deoxygalactonojirimycn to the individual, wherein the 1-deoxygalactonojirimycin binds to alpha-galactosidase A in an amount effective to increase activity of the alpha-galactosidase A. The present invention also provides a method for monitoring and increasing a therapeutic response of a patient with Fabry disease following administration of a specific pharmacological chaperone of α-galactosidase A by evaluating the effect on the cytoplasmic staining pattern of a cell from the patient, wherein detection of a staining pattern in the cell that is similar to the staining pattern in a cell from a healthy individual indicates that the individual with Fabry disease is a responder.

### CROSS-REFERENCE TO RELATED APPLICATIONS

This application claims priority to U.S. Provisional Application Ser. No. 60/909,185, filed Mar. 30, 2007, which is hereby incorporated by reference in its entirety herein.

### FIELD OF THE INVENTION

The present invention provides a method for treating Fabry disease by restoring α-galactosidase A activity by at least 3-fold, and more particularly by restoring its activity to levels in the normal range. In addition, the present invention provides a method for monitoring the treatment of an individual having Fabry disease with a specific pharmacological chaperone by evaluating changes in the presence and/or quantity of specific surrogate markers. The present invention also provides a method for monitoring the treatment of an individual having Fabry disease with a specific pharmacological chaperone by evaluating the effects of treatment at the sub-cellular level.

### BACKGROUND

Fabry disease is a glycosphingolipid (GSL) storage disease caused by an X-linked inherited deficiency of lysosomal α-galactosidase A (α-Gal A), an enzyme responsible for the hydrolysis of terminal α-galactosyl residues from glycosphingolipids. A deficiency in the enzyme activity results in a progressive deposition of neutral glycosphingolipids, predominantly globotriaosylceramide (ceramide trihexoside, CTH, GL-3), in vascular endothelial cells causing renal failure along with premature myocardial infarction and strokes in patients with this condition. This disorder is classified by clinical manifestations into two groups: a classic form with generalized vasculopathy, and an atypical variant form, with clinical manifestations limited to cardiac tissue.

The frequency of the disease is estimated to be about 1:40,000 in males, and is reported throughout the world within different ethnic groups. In classically affected males, the clinical manifestations include angiokeratoma (small, raised reddish-purple blemishes on the skin), acroparesthesias (burning in hands and feet), hypohidrosis (decreased ability to sweat), and characteristic corneal and lenticular opacities (The Metabolic and Molecular Bases of Inherited Disease, 8th Edition 2001, Scriver et al., ed., pp. 3733-3774, McGraw-Hill, New York). Lipid storage may lead to impaired arterial circulation and increased risk of heart attack or stroke. The heart may also become enlarged and the kidneys may become progressively involved. Other symptoms include fever and gastrointestinal difficulties, particularly after eating. Some female carriers may also exhibit symptoms.

The affected male's life expectancy is reduced, and death usually occurs in the fourth or fifth decade as a result of vascular disease of the heart, brain, and/or kidneys. In contrast, patients with the milder “cardiac variant” normally have 5-15% of normal α-Gal A activity, and present with left ventricular hypertrophy or a cardiomyopathy. These cardiac variant patients remain essentially asymptomatic when their classically affected counterparts are severely compromised. Recently, cardiac variants were found in 11% of adult male patients with unexplained left ventricular hypertrophic cardiomyopathy, suggesting that Fabry disease may be more frequent than previously estimated (Nakao et al., N. Engl. J. Med. 1995; 333: 288-293).

The α-Gal A gene has been mapped to Xq22, (Bishop et al., Am. J. Hum. Genet. 1985; 37: A144), and the full-length cDNA and entire 12-kb genomic sequences encoding α-Gal A have been reported (Calhoun et al., Proc. Natl. Acad. Sci. USA. 1985; 82: 7364-7368; Bishop et al., Proc. Natl. Acad. Sci. USA. 1986; 83: 4859-4863; Tsuji et al., Eur. J. Biochem. 1987; 165: 275-280; and Kornreich et al., Nucleic Acids Res. 1989; 17: 3301-3302). There is a marked genetic heterogeneity of mutations that cause Fabry disease (The Metabolic and Molecular Bases of Inherited Disease, 8th Edition 2001, Scriver et al., ed., pp. 3733-3774, McGraw-Hill, New York.; Eng et al., Am. J. Hum. Genet. 1993; 53: 1186-1197; Eng et al., Mol. Med. 1997; 3: 174-182; and Davies et al., Eur. J. Hum. Genet. 1996; 4: 219-224). To date, a variety of missense, nonsense, and splicing mutations, in addition to small deletions and insertions, and larger gene rearrangements have been reported.

### Treatment

Treatment of Fabry disease is predominantly by enzyme replacement therapy (ERT) with recombinant α-Gal A, e.g., with the product marketed as Fabrazyme® (Genzyme, Inc.) and Replagal® (TKT, Inc.). ERT typically involves intravenous, subcutaneous or intramuscular infusion of a purified form of the corresponding wild-type protein, or implantation of the protein in a bio-erodable solid form for extended-release. One of the main complications with ERT is attainment and maintenance of therapeutically effective amounts of protein due to rapid degradation of the infused protein. As a result, ERT requires numerous, high-dose infusions and as a result, is costly and time consuming.

ERT has several additional drawbacks, such as difficulties with large-scale generation, purification and storage of properly folded protein, obtaining glycosylated native protein, generation of an anti-protein immune response in some patients, and failure of protein to cross the blood-brain barrier in sufficient quantities to affect diseases having significant central nervous system involvement.

Gene therapy using recombinant vectors containing nucleic acid sequences that encode a functional protein, or genetically modified human cells that express a functional protein, is also being used to treat protein deficiencies and other disorders that benefit from protein replacement. Although promising, this approach is also limited by technical difficulties such as the inability of vectors to infect or transduce dividing cells, low expression of the target gene, and regulation of expression once the gene is delivered (e.g., many viral vectors require cells to be dividing for efficacy).

A third, relatively recent approach to treating protein deficiencies involves the use of small molecule inhibitors to inhibit synthesis the natural substrate of the deficient enzyme protein, thereby ameliorating the pathology. This “substrate deprivation” approach has been specifically described for a class of about 40 related enzyme disorders called lysosomal storage disorders or glycosphingolipid storage disorders. These heritable disorders are characterized by deficiencies in lysosomal enzymes that catalyze the breakdown of glycolipids in cells, resulting in an abnormal accumulation of lipids, which disrupts cellular function. The small molecule inhibitors proposed for use as therapy are specific for inhibiting the enzymes involved in synthesis of glycolipids, reducing the amount of cellular glycolipid that needs to be broken down by the deficient enzyme. This approach is also limited in that glycolipids are necessary for biological function, and excess deprivation may cause adverse effects. Specifically, glycolipids are used by the brain to send signals from one neuron to another. If there are too few or too many glycolipids, the ability of the neuron to send signals is impeded.

In addition, treatment with one substrate inhibitor, NB-DNJ, for lysosomal storage disease Gaucher disease is associated with numerous adverse events in humans, including peripheral neuropathy and severe gastrointestinal effects. These effects present even at low doses of 150 mg/day, which is not a therapeutically effective dose. In view of the foregoing, administration of NB-DNJ for the treatment of Gaucher in the U.S. and Europe is very limited to adults with mild-to-moderate type I Gaucher patients where ERT is not an option, and is not approved for therapy in Canada (Weinreb et al., Am J. Hematology. 2005. 80: 223-29).

A fourth approach, a specific chaperone strategy, rescues mutated proteins from degradation presumably in the endoplasmic reticulum (ER) or in other cellular protein degradation/disposal systems. Previous patents and publications describe a therapeutic strategy for rescuing endogenous enzyme proteins, including misfolded lysosomal enzymes, from degradation by the ER quality control machinery. In particular embodiments, this strategy employs small molecule reversible inhibitors which specifically bind to a defective lysosomal enzyme associated with a particular lysosomal disorder. In the absence of therapy, the mutated enzyme protein folds improperly in the ER (Ishii et al., Biochem. Biophys. Res. Comm. 1996; 220: 812-815), is retarded in its maturation to a final product, and is subsequently degraded in the ER. The chaperone strategy involves the use of a compound that facilitates the correct folding of a mutated protein, to prevent undue or abnormal degradation from the ER quality control system, or accumulation of misfolded protein in the cell. These specific chaperones are designated pharmacological chaperones (or active site-specific chaperones).

The chaperone strategy has been described and exemplified for enzymes involved in lysosomal storage disorders as in U.S. Pat. Nos. 6,274,597, 6,583,158, 6,589,964, 6,599,919, and 6,916,829 to Fan et al., which are incorporated herein by reference in their entirety. For example, a small molecule derivative of galactose, 1-deoxygalactonojirimycin (DGJ), a potent competitive inhibitor of the mutant Fabry enzyme α-galactosidase A (α-Gal A), effectively increased in vitro stability of the human mutant α-Gal A (R301Q) at neutral pH, and it enhanced the mutant enzyme activity in lymphoblasts established from Fabry patients with R301Q or Q279E mutations by 45% or more. Furthermore, oral administration of DGJ to transgenic mice overexpressing a mutant (R301Q) α-Gal A substantially elevated the enzyme activity in major organs (Fan et al., Nature Med. 1999; 5: 112-115). Similar rescue of acid β-glucosidase (glucocerebrosidase; Gba) from Gaucher patient cells in tissue culture has been described using another iminosugar, isofagomine (IFG), and its derivatives, described in pending U.S. Pat. No. 6,583,158 to Fan et al., and using other compounds specific for Gba (described in pending U.S. patent application Ser. Nos. 10/988,428, and 10/988,427, both filed Nov. 12, 2004).

Successful rescue of a misfolded protein by a chaperone that is an inhibitor depends on achieving a concentration of the specific inhibitor in vivo that is lower than necessary to completely inhibit the enzyme, in contrast to the substrate deprivation approach in which enzyme inhibitory concentrations are required. The low dose of chaperone that will be effective also reduces the amount and severity of any adverse side effects that plagues the use of substrate inhibitors. However, to date the clinical and preclinical studies on these approaches to treating Fabry disease suggest that the improvement, though beneficial, cannot bring the patient to normal levels of α-Gal A activity. Furthermore, the effects of clinical treatment of Fabry disease using a pharmacological chaperone on surrogate markers or at the subcellular level remain unknown.

### BRIEF DESCRIPTION OF THE DRAWINGS

FIG. 1. FIG. 1 depicts α-Gal A enzymatic activity data over time for placebo, 50 mg b.i.d. and 150 mg b.i.d. dosages in healthy volunteers.

FIG. 2. FIG. 2 depicts the average increases in white blood cell activity in Fabry patients as a result of DGJ administration over 48 weeks.

FIG. 3. FIG. 3 depicts GL-3 levels in two Fabry patients following treatment with DGJ for 12 weeks.

### SUMMARY OF THE INVENTION

The present invention provides a method of treating and method of monitoring the response to treatment of Fabry patients with specific pharmacological chaperones such as DGJ by evaluating changes in the presence and/or levels of surrogate markers associated with Fabry disease.

In one embodiment, the method of treatment involves determining whether there is an improvement of a surrogate marker that is associated with Fabry disease following administration of a specific pharmacological chaperone of α-galactosidase A.

In a specific embodiment, an improvement indicates that the patient is a responder.

In one embodiment, the surrogate marker is a systemic surrogate marker. In particular embodiments, the marker is lysosomal α-galactosidase A activity in cells and tissue or GL-3 accumulation. In another embodiment, the surrogate marker is a sub-cellular surrogate marker selected from aberrant trafficking of α-galactosidase A in cells from Fabry patients from the ER to the lysosome; aberrant trafficking of cellular lipids though the endosomal pathway; the presence of increased amounts misfolded α-galactosidase A in the ER or cytosol; the presence of cellular stress resulting from toxic accumulation of α-galactosidase A (as determined by gene and/or protein expression of stress-related markers); aberrant endosomal pH levels; aberrant cell morphology; suppression of the ubiquitin/proteasome pathway; and an increase in the amount of ubiquitinated proteins.

In a specific embodiment, the pharmacological chaperone is an inhibitor of α-galactosidase A. In another embodiment, the inhibitor is a reversible competitive inhibitor, such as 1-deoxygalactonojirimycin.

The present invention also provides for a method for monitoring a therapeutic response of a patient with Fabry disease following administration of a specific pharmacological chaperone of α-galactosidase A. This method includes evaluating the effect on the cytoplasmic staining pattern of a cell from the patient, wherein detection of a staining pattern in the cell that is similar to the staining pattern in a cell from a healthy individual indicates that the individual with Fabry disease is a responder.

In one embodiment, cytoplasmic staining is lysosomal staining. In other embodiments, the lysosomal staining is detection of the presence of α-galactosidase A, LAMP-1 expression, or polyubiquitinated proteins.

In a specific embodiment, the pharmacological chaperone is an inhibitor of α-galactosidase A. In another embodiment, the inhibitor is a reversible competitive inhibitor, such as 1-deoxygalactonojirimycin.

The present invention also provides a method for increasing the activity of α-galactosidase A protein in an individual in need thereof This method includes administering to the individual an effective amount of a specific pharmacological chaperone that binds to the protein in an amount effective to increase activity of the protein in the individual by at least about 50%.

In a particular embodiment, the α-galactosidase A protein is a wild type protein. In another embodiment, α-galactosidase A protein is an enzyme. In one embodiment, the enzyme is a lysosomal enzyme. In yet another embodiment, the lysosomal enzyme is a wild type α-galactosidase A protein.

In one embodiment of this invention, the pharmacological chaperone is an inhibitor of α-galactosidase A. In another embodiment, the inhibitor is a reversible competitive inhibitor, such as 1-deoxygalactonojirimycin.

In one embodiment, the 1-deoxygalactonojirimycin is administered in an amount effective to increase the activity of the protein by at least about 50%, preferably by at least 2-fold (about 100%), more preferably at least about 3-fold to 5-fold, more preferably at least about 10-fold, and even more preferably at least about 15-fold.

In one embodiment of the invention, the individual is homozygous for the wild type protein. In another embodiment, the individual is heterozygous for the wild type protein and has a mutant genotype for the other allele encoding the protein. In paritcular embodiments, the individual suffers from Fabry disease.

In another embodiment of the invention, the α-galactosidase A activity increases by at least 2-fold, preferably at least 3-fold to 5-fold, more preferably at least 10-fold. In a specific embodiment, the α-galactosidase A activity increases to within a normal range.

The present invention also provides for a method of treating Fabry disease in a patient in need thereof, wherein the method includes administering to the individual an effective amount of 1-deoxygalactonojirimycn, wherein the 1-deoxygalactonojirimycn binds to alpha-galactosidase A in an amount effective to increase activity of the alpha-galactosidase A in the patient by at least about 2-fold, preferably at least 3-fold to 5-fold, more preferably at least 10-fold. In a specific embodiment, the α-galactosidase A activity increases to within a normal range.

### DETAILED DESCRIPTION

The present invention provides for titrating a dose of a specific pharmacological chaperone to achieve at least about a 3-fold increase in the level of enzyme activity, or increase the level of enzyme activity to unprecedented, normal, levels. In specific embodiments, the specific pharmacological chaperone is a reversible competitive inhibitor of α-Gal A, preferably DGJ.

In addition, the present invention provides method for monitoring a patient's response to treatment of Fabry disease with a specific pharmacological chaperone that is a reversible competitive inhibitor of α-Gal A, such as 1-DGJ. In particular, treatment is monitored by assaying biological samples for markers associated with Fabry disease, including but not limited to cellular deposits of globotriaosylceramide (GL-3) in cells, including cells within blood vessel walls, and kidney, heart, and skin cells; urinary GL-3 levels; α-Gal A activity in leukocytes, plasma, skin, kidney, and heart cells; and urinary proteinuria. Additional markers include the presence of angiokeratomas, decreased sweating, painful tingling or burning sensations in the extremities, gastrointestinal disturbances, cataracts or other visual impairment, and hearing loss.

In addition, evaluation of sub-cellular markers of disease, such as trafficking of mutant protein and alleviation of cell stress due to toxic accumulation of mutant proteins is also contemplated.

The invention is based, in part, on results obtained from Phase I and II tests of DGJ for treatment of Fabry disease. Among the discoveries that were made were identification of changes to surrogate markers of Fabry disease indicative of a therapeutic effect of the therapy; identification of a dosage range and dosage regimen that increase α-Gal A activity; and, surprisingly, that DGJ treatment could be titrated to achieve at least a 2-fold, and more preferably a 3-fold to 5-fold, increase in α-Gal A activity, and even up to 10-fold and 15-fold in some instances, e.g., as measured in white blood cells. In addition, administration of DGJ could be titrated to restore α-Gal A activity levels, as measured in white blood cells, to the normal range, an unprecedented therapeutic effect.

### Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “Fabry disease” refers to an X-linked inborn error of glycosphingolipid catabolism due to deficient lysosomal α-galactosidase activity. This defect causes accumulation of globotriosylceramide and related glycosphingolipids in vascular endothelial lysosomes of the heart, kidneys, skin, and other tissues.

The term “atypical Fabry disease” refers to patients with primarily cardiac manifestations of the α-Gal A deficiency, namely progressive globotriaosylceramide accumulation in myocardial cells that leads to significant enlargement of the heart, particularly the left ventricle.

A “Fabry disease patient” refers to an individual who has been diagnosed with Fabry disease due to a mutated α-galactosidase A as defined further below.

Human α-galactosidase A refers to an enzyme encoded by the human Gla gene which has an amino sequence as set forth in SEQ ID NO: 2. The human α-Gal A enzyme consists of 429 amino acids and is in GenBank Accession No. U78027.

As used herein the term “mutant α-Gal A” refers to an α-Gal A which has a mutation which results in the inability of the enzyme to achieve its native conformation under the conditions normally present in the ER. The failure to achieve this conformation results in the enzyme being degraded, rather than being transported through their normal pathway in the protein transport system to the lysosome. This term also encompasses an other α-Gal A mutations that result in decreased enzyme activity or a more rapid turnover.

Exemplary α-Gal A mutations associated with Fabry disease include R301Q, L166V, A156V, G272S, and M2961.

As used herein, the term “specific pharmacological chaperone” (“SPC”) or “pharmacological chaperone” refers to any molecule including a small molecule, protein, peptide, nucleic acid, carbohydrate, etc. that specifically binds to a protein and has one or more of the following effects: (i) enhancing the formation of a stable molecular conformation of the protein; (ii) inducing trafficking of the protein from the ER to another cellular location, preferably a native cellular location, i.e., preventing ER-associated degradation of the protein; (iii) preventing aggregation of misfolded proteins; and/or (iv) restoring or enhancing at least partial wild-type function and/or activity to the protein. A compound that specifically binds to e.g., α-Gal A, means that it binds to and exerts a chaperone effect on α-Gal A and not a generic group of related or unrelated enzymes. In the present invention, the SPC may be a reversible competitive inhibitor. Following is a description of some specific pharmacological chaperones contemplated by this invention:

1-deoxygalactonojirimycin refers to a compound having the following structures:

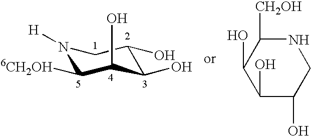

This term includes both the free base and any salt forms. The hydrochloride salt of DGJ is known as migalastat hydrochloride (Miglustat).

Still other SPCs for α-Gal A are described in U.S. Pat. Nos. 6,274,597, 6,774,135, and 6,599,919 to Fan et al., and include α-3,4-di-epi-homonojirimycin, 4-epi-fagomine, and α-allo-homonojirimycin, N-methyl-deoxygalactonojirimycin, β-1-C-butyl-deoxygalactonojirimycin, and α-galacto-homonojirimycin, calystegine A₃, calystegine B₂, N-methyl-calystegine A₃, and N-methyl-calystegine B₂.

A “surrogate marker” or “surrogate clinical marker” of Fabry disease refers to the abnormal presence of, increased levels of, abnormal absence of, or decreased levels of a biomarker or symptom that is associated with Fabry disease (but is not associated with a healthy individual), and which is a reliable indicator of Fabry disease either alone or in combination with other abnormal markers or symptoms of Fabry disease.

As non-limiting examples, surrogate markers of Fabry disease include decreased lysosomal α-Gal A activity in cells (e.g., fibroblasts) and tissue; cellular deposition of GL-3; increased plasma concentrations of homocysteine and vascular cell adhesion molecule-1 (VCAM-1); GL-3 accumulation within myocardial cells and valvular fibrocytes, leading to cardiac hypertrophy (especially of the left ventricle), valvular insufficiency, and arrhythmias; proteinuria; increased urinary concentrations of lipids such as CTH, lactosylceramide, ceramide, and decreased urinary concentrations of glucosylceramide and sphingomyelin (Fuller et al., Clinical Chemistry. 2005; 51: 688-694); the presence of laminated inclusion bodies (Zebra bodies) in glomerular epithelial cells; renal failure; hypohidrosis (which causes heat intolerance); the presence of angiokeratomas; and hearing abnormalities such as high frequency sensorineural hearing loss progressive hearing loss, sudden deafness, or tinnitus. Neurological symptoms include transient ischemic attack (TIA) or stroke; and neuropathic pain manifesting itself as acroparaesthesia (burning or tingling in extremities).

Characteristic markers of Fabry disease can occur in male hemizygotes and female heterozygotes (carriers) with the same prevalence, although females typically are less severely affected.

Other surrogate markers are present at the sub-cellular level (“sub-cellular surrogate markers”) and include aberrant trafficking of α-Gal A in cells from Fabry patients from the ER to the lysosome; aberrant trafficking of lipids though the endosomal pathway; the presence of increased amounts misfolded α-Gal A in the ER or cytosol; the presence of cellular stress resulting from toxic accumulation of α-Gal A (as determined by gene and/or protein expression of stress-related markers); aberrant endosomal pH levels; aberrant cell morphology; suppression of the ubiquitin/proteasome pathway; or an increase in the amount of ubiquitinated proteins.

An “an improvement in a surrogate marker” refers to an effect, following treatment with an SPC of the amelioration, reduction, or increase in of one or more clinical surrogate markers which are abnormally present, abnormally absent, or present in increased or decreased quantities in Fabry disease relative to a healthy individual who does not have Fabry disease and who does not have an other disease that accounts for the abnormal presence, absence, or altered quantities of that surrogate marker.

A “responder” is an individual diagnosed with a disease associated with Fabry disease and an associated a α-Gal A mutation and treated and monitored according to the presently claimed method, who exhibits an improvement in one or more surrogate markers, and/or amelioration of, or reversal of, disease progression.

In addition, a determination whether an individual is a responder can be made at the sub-cellular level by evaluating, e.g., intracellular trafficking of the mutant α-Gal A protein in response to treatment with an SPC. Restoration of trafficking from the ER to the lysosome is indicative of a response. Other sub-cellular evaluations that can be assessed to determine if an individual is a responder include improvements in the above-referenced sub-cellular surrogate markers.

In a specific embodiment, the invention provides for a method of treatment with DGJ to achieve about a 3-fold increase in α-Gal A activity, e.g., as determined in white blood cells from the patient. In a further embodiment, the invention provides a method of treatment with DGJ to achieve at least a 5-fold increase in α-Gal A activity, or a 10-fold increase in α-Gal A activity, and in specific embodiments, a 15-fold increase in α-Gal A activity, e.g., as measured in white blood cells. The invention minimally provides for at least a 2-fold increase in α-Gal A activity.

Similarly, the invention provides for achieving an increase in α-Gal A activity in white blood cells from Fabry patients to within the normal range, a result that has not been achieved with other therapeutic strategies, and which the preclinical data could not have predicted would be the case. In a specific embodiment, the “normal range” is the range of activity as described in data from The Metabolic & Molecular Bases of Inherited Disease, 8th Edition McGraw Hill, 2001).

The terms “therapeutically effective dose” and “effective amount” refer to the amount of the specific pharmacological chaperone that is sufficient to result in a therapeutic response. A therapeutic response may be any response that a user (e.g., a clinician) will recognize as an effective response to the therapy, including improvements in any of the foregoing symptoms and surrogate clinical markers. A therapeutic response may be any response that a user (e.g., a clinician) will recognize as an effective response to the therapy, including improvements in the foregoing symptoms and surrogate clinical markers. Thus, a therapeutic response will generally be an amelioration of one or more symptoms or markers of a disease or disorder, such as those described above.

The phrase “pharmaceutically acceptable” refers to molecular entities and compositions that are physiologically tolerable and do not typically produce untoward reactions when administered to a human. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans. The term “carrier” refers to a diluent, adjuvant, excipient, or vehicle with which the compound is administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Numerical quantities given herein are approximate unless stated otherwise, meaning that the term “about” or “approximately” can be inferred when not expressly stated.

### Formulations and Administration

Any appropriate pharmaceutical formulation may be used to deliver the SPC systemically. In certain embodiments, the SPC is a reversible competitive inhibitor. In one embodiment, the inhibitor is an imino sugar, e.g., DGJ, which is administered as monotherapy, preferably in an oral dosage form. In this embodiment, it is contemplated that the dosing regimen should be one that provides a constant, steady state level of compound in the plasma of the individual being treated. This can be obtained either by daily administration in divided doses, or controlled-release formulations, or by less frequent administration of sustained-release dosage forms.

### Formulations

The SPC, can be administered in a form suitable for any route of administration, including e.g., orally in the form tablets, capsules, or liquid, or in sterile aqueous solution for injection. In certain embodiments, the SPC is an inhibitor, preferably a reversible competitive inhibitor such as a DGJ sugar. When the compound is formulated for oral administration, the tablets or capsules can be prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art.

In a specific embodiment, DGJ-HCl is administered as a powder-filled, hard, gelatin capsule with magnesium stearate (vegetable) and starch 1500 (25 mg DGJ/capsule).

Liquid preparations for oral administration may take the form of, for example, solutions, syrups or suspensions, or they may be presented as a dry product for constitution with water or another suitable vehicle before use. Such liquid preparations may be prepared by conventional means with pharmaceutically acceptable additives such as suspending agents (e.g., water, sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled or sustained release of the ceramide-specific glucosyltransferase inhibitor.

In another specific embodiment, 100 mg of DGJ is administered in an oral solution in water (about 240 ml).

The pharmaceutical formulations of the SPC suitable for parenteral/injectable use generally include sterile aqueous solutions, or dispersions and sterile powders for the extemporaneous preparation of sterile injectable solutions or dispersion. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be brought about by various antibacterial and antifungal agents, for example, parabens, chlorobutanol, phenol, benzyl alcohol, sorbic acid, and the like. In many cases, it will be reasonable to include isotonic agents, for example, sugars or sodium chloride. Prolonged absorption of the injectable compositions can be brought about by the use in the compositions of agents delaying absorption, for example, aluminum monosterate and gelatin.

Sterile injectable solutions are prepared by incorporating the SPC in the required amount in the appropriate solvent with various of the other ingredients enumerated above, as required, followed by filter or terminal sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

The above formulations can contain an excipient or excipients. Pharmaceutically acceptable excipients which may be included in the formulation are buffers such as citrate buffer, phosphate buffer, acetate buffer, and bicarbonate buffer, amino acids, urea, alcohols, ascorbic acid, phospholipids, proteins, such as serum albumin, collagen, and gelatin; salts such as EDTA or EGTA, and sodium chloride; liposomes; polyvinylpyrollidone; sugars such as dextran, mannitol, sorbitol, and glycerol; propylene glycol and polyethylene glycol (e.g., PEG-4000, PEG-6000); glycerol, glycine or other amino acids and lipids. Buffer systems for use with the formulations include citrate, acetate, bicarbonate, and phosphate buffers. Phosphate buffer is a preferred embodiment.

The formulations can also contain a non-ionic detergent. Preferred non-ionic detergents include Polysorbate 20, Polysorbate 80, Triton X-100, Triton X-114, Nonidet P-40, Octyl α-glucoside, Octyl β-glucoside, Brij 35, Pluronic, and Tween 20.

### Administration

The route of administration of the SPC may be oral (preferably) or parenteral, including intravenous, subcutaneous, intra-arterial, intraperitoneal, ophthalmic, intramuscular, buccal, rectal, vaginal, intraorbital, intracerebral, intradermal, intracranial, intraspinal, intraventricular, intrathecal, intracisternal, intracapsular, intrapulmonary, intranasal, transmucosal, transdermal, or via inhalation.

Administration of the above-described parenteral formulations of the SPC may be by periodic injections of a bolus of the preparation, or may be administered by intravenous or intraperitoneal administration from a reservoir which is external (e.g., an i.v. bag) or internal (e.g., a bioerodable implant). See, e.g., U.S. Pat. Nos. 4,407,957 and 5,798,113, each incorporated herein by reference. Intrapulmonary delivery methods and apparatus are described, for example, in U.S. Pat. Nos. 5,654,007, 5,780,014, and 5,814,607, each incorporated herein by reference. Other useful parenteral delivery systems include ethylene-vinyl acetate copolymer particles, osmotic pumps, implantable infusion systems, pump delivery, encapsulated cell delivery, liposomal delivery, needle-delivered injection, needle-less injection, nebulizer, aeorosolizer, electroporation, and transdermal patch. Needle-less injector devices are described in U.S. Pat. Nos. 5,879,327; 5,520,639; 5,846,233 and 5,704,911, the specifications of which are herein incorporated by reference. Any of the formulations described above can be administered using these methods.

Furthermore, a variety of devices designed for patient convenience, such as refillable injection pens and needle-less injection devices, may be used with the formulations of the present invention as discussed herein.

### Dosage

Persons skilled in the art will understand that an effective amount of the compounds used in the methods of the invention can be determined by routine experimentation, but is expected to be an amount resulting in serum levels between 0.01 and 100 μM, preferably between 0.01 and 10 μM, most preferably between 0.05 and 1 μM. Clinical studies, and preclinical studies in mice, suggest that the pharmacological effect (enzyme activity enhancement) of DGJ is seen at a plasma concentration of approximately 0.4 μM.

The effective dose of the SPC is expected to be between 0.5 and 1000 mg/kg body weight per day. In specific embodiments where the SPC is DGJ, the dose is between about 10-600 mg/day, more specifically 25-300 mg/day, more specifically, 50-150 mg/day.

In particular embodiments, DGJ is administered at 25 mg b.i.d., 100 mg b.i.d. or 250 b.i.d. Data from the multiple-dose phase 1 study indicate that a trough level of 0.4 μM is obtained with dosing of 50 mg b.i.d. Data from a phase II study on Fabry patients demonstrated increased enzyme activity at the lowest dose evaluated (25 mg b.i.d.).

### Fabry Disease Treatment Monitoring Using Surrogate Markers

The present invention also provides a method for monitoring the treatment of Fabry patients with specific pharmacological chaperones. Specifically, various assays are employed to evaluate the progress of the disease and its response to treatment with DGJ. In particular, various systemic and sub-cellular markers can be assayed. The monitoring aspect of the present invention encompasses both invasive and non-invasive measurement of various cellular substances.

Globotriaosylceramide accumulation. A method for measuring globotriaosylceramide (GB₃, or GL-3) levels in plasma and urine of humans affected by Fabry disease is described in, e.g., Boscaro et al., Rapid Commun Mass Spectrom. 2002; 16(16):1507-14. In this reference, the analyses are performed using flow injection analysis-electrospray ionization-tandem mass spectrometry (FIA-ESI-MS/MS).

Immunoelectron-microscopic detection of GL-3 accumulated in the skin of patients with Fabry disease has been recently described in Kanekura et al., Br J Dermatol. 2005; 153(3):544-8. This method is sensitive enough to detect lysosomal accumulation of GL-3. Skin biopsies can be obtained by using a “punch” device, which removes a sample layer of skin.

Renal biopsies are performed using ultrasound, x-ray or CT scan guidance. Under some circumstances, the biopsy is be performed by running the biopsy catheter through one of the neck veins-this is called a trans jugular biopsy. GL-3 accumulation in kidney, specifically in all renal cell types, including vascular endothelial cells, vascular smooth muscle cells, mesangial cells and interstitial cells, podocytes and distal tubular epithelial cells, had been described in Thurburg et al., Kidney Int. 2002; 62(6):1933-46. Ultrastructural study (electron microscopy) of kidney biopsies can reveal typical inclusion bodies in the cytoplasm of all types of renal cells (Sessa et al., J Inherit Metab Dis. 2001; 24 Suppl 2:66-70). The cells are characterized by concentric lamellation of clear and dark layers (“zebra” or “onion-skin” appearance) with a periodicity of 35-50 A.

Kidney function can be assessed by determining glomerular filtration rate (ml/min) and by assessing serum creatine levels according to well-established methods. Other renal assessments include 24-hour protein excretion, urine protein electrophoresis, total protein, microalbumin, urine beta-2 microglobulin titers. Reduction in GL-3 sediment and proteinuria is a direct measurement of renal health.

Recently, atmospheric pressure photoionization mass spectrometry (APPI-MS) was shown to be an efficient method for the analysis of GL-3 molecular species, both in direct injection and by coupling with liquid chromatography (LC). This technique allowed the detection of a great number of species from biological samples isolated from Fabry patients (Delobel et al., J Mass Spectrom. 2005 Nov. 14; [Epub ahead of print]).

α-Galactosidase activity. As indicated above, non-invasive assessment of α-Gal A activity can be measured in blood leukocytes or in cultured fibroblasts from skin biopsies. Such assays typically involve extraction of blood leukocytes from the patient, lysing of the cells, and determining the activity in the lysate upon addition of an enzyme substrate such as 4-methyl umbelliferal alpha-D-galactoside an/or N-acetylgalactosamine (see U.S. Pat. No. 6.274,597).

Two sensitive immunoassays for the measurement of α-Gal A activity and protein to determine the concentrations of alpha-galactosidase in blood and plasma are described in Fuller et al., Clin Chem. 2004; 50(11):1979-85.

According to the present invention, it is possible to titrate dosage to achieve at least a 2-fold increase in α-Gal A activity, and in some cases at least a 3-fold increase in α-Gal A activity, at least 5-fold, up to 10-fold, and in some embodiments, a 15-fold increase. These results can be observed with white blood cells.

In another embodiment, the invention provides for titrating a dosage of DGJ to achieve normal levels of α-Gal A activity in a Fabry patient.

Cardiac evaluation. Increases in alpha-Gal A activity may play a role monitoring or detecting heart disease or in at least a subset of heart disease patients. Evaluation of GL-3 in cardiac cells can be achieved through endomyocardial biopsies. This is an invasive procedure that involves using a bioptome (a small catheter with a grasping device on the end) to obtain a small piece of heart muscle tissue.

GL-3 present in perinuclear vacuoles will stain positive with an acid stain. In addition, histological examination of the biopsies can be done using transmission electron microscopy to ascertain thickening of endocardium to measure ventricular mass, or to determine the presence of hypertrophic myocardial fibers.

In addition, common carotid and radial artery diameter, intima-media thickness (IMT) and distensibility have been assessed using high-definition echotracking systems and aplanation tonometry. (Boutouyrie et al., Acta Paediatr Suppl. 2002; 91(439):62-6. Cardiac myocytes will also be examined for accumulation of GL-3. Macroscopic cardiac morphology can be assessed using MRI or Doppler echocardiography. Cardiac function can be assessed by, e.g., determining left ventricular ejection fraction and using electrocardiograms.

Neuropathic pain/peripheral neuropathy. Pain in the extremities can be assessed using subjective tests given to the patient. In addition, to evaluate neuropathy, Quantitative Sensory Testing (CASE study) can be used. A CASE study is a biophysical technique in which a patient is asked to push a button as soon as he feels either a sensation of cold, warmth, or vibration. These stimulations are delivered by an electrode that is put on the skin of the hand or foot.

Cerebrovascular. In addition to stroke and hypertension, other Fabry-related cerebrovascular signs and symptoms associated can include hemiparesis, vertigo, double vision; seizures; basilar artery ischema and aneurism; labyrinthine disorders or cerebral hemorrhage.

Neurological. In male and female patients with Fabry disease, significant age-related cerebral white matter lesions (WMLs) can be found. Evaluation of neurological effects can be assessed using, e.g., Quantitative Sudomotor Axon Reflex Test (QSART). QSART is a routine test of autonomic function and a sensitive test in distal small-fiber neuropathy such as observed in Fabry disease.

Hypohydrosis/anhidrosis. Impaired sweating and heat intolerance in Fabry patients has been attributed to selective peripheral nerve damage or to intracytoplasmic lipid deposits in the small blood vessels surrounding sweat glands.

Hilz et al. (i Acta Paediatr Suppl. 2002; 91(439):38-42) have described the methods to assess impairment of temperature perception, vibratory perception, sudomotor and eccrine sweat gland function, and limb and superficial skin blood flow and vasoreactivity in Fabry patients. These methods include thermal provocation tests, quantitative sudomotor axon reflex testing (QSART) and venous occlusion plethsmography. QSART has three parts and measures resting skin temperature, resting sweat output, and stimulated sweat output. Measurements are typically taken on arms, legs or both. A small plastic cup is placed on the skin and the temperature and amounts of sweat under the skin are measured. To stimulate sweat a chemical is delivered electrically through the skin to a sweat gland, but the patient will only feel warmth. A computer is used to analyze the data to determine how well the nerves and sweat glands are functioning.

In addition, a reduction of tears and saliva is also observed in about 40% of Fabry disease patients.

Temperature intolerance. In addition to heat intolerance, cold and heat sensitivity often results from lipid deposition in small vessel walls, perineural cells, and unmyelinated or myelinated nerve cells resulting in small fiber neuropathy.

Ophthalmologic opacities. Fabry disease patients almost universally exhibit whorled corneal opacities, lenticular opacities, and vascular lesions of the conjunctivae and retina. Corneal opacities can be seen using slit lamp microscopy. Two types of lens opacities have been noted in Fabry patients: cream-colored anterior capsular deposits in the lens (sometimes distributed like a propeller), and whitish, granular spoke-like deposits on the posterior lens (referred to as Fabry cataracts).

Hearing loss. Non-invasive methods to evaluate cochlear functions using conventional audiometry, tympanometry, ABR audiometry, and otoacoustic emissions is described in Germain et al., BMC Med Genet. 2002; 3(1):10).

Gastrointestinal disturbance. Gastrointestinal symptoms may result from deposition of glycosphingolipids in mesenteric blood vessels and autonomic ganglia. Symptoms include postprandial bloating; abdominal cramping and pain; early satiety; diarrhea; constipation; nausea; vomiting.

Other surrogate markers. Other markers of Fabry disease include Lymphoedema (swelling of the extremities) due to accumulation of GL-3. In addition, it was recently discovered that there was a signififcant decrease in diastolic blood pressure in patients with Fabry disease, which may account for exercise tolerance (Bierer et al., Respiration. 2005; 72(5):504-11).

It is to be understood that these markers can be used to monitor treatment only if they are identified to be abnormal prior to treatment. In addition, it is understood that the abnormal elevation of the markers be correlated with the presence of Fabry disease, and not attributed to other causes or concomitant diseases such as kidney disease or other cerebrovascular disease.

### Molecular Biology Monitoring Assays to Detect Sub-Cellular Markers

Monitoring of treatment of Fabry disease with specific pharmacological chaperones can be done at the subcellular level in addition to the systemic or macroscopic level, described above. For example, disturbances in endosomal-lysosomal membrane trafficking of lipids to the Golgi complex are a characteristic of lysosomal storage disease (Sillence et al., J Lipid Res. 2002; 43(11):1837-45). Accordingly, one way of monitoring treatment of Fabry would be to contact cells from patients with labeled lipid (BODIPY-cholesterol) and monitor its trafficking in endosomal structures. Pathological accumulation in endosomal structures, for example, would be indicative that the patient is not responding well to treatment.

As one example, pH-sensitive fluorescent probes that are endocytosed by the cells can be used to measure pH ranges in the lysosomes and endosomes (i.e. fluorescein is red at pH 5, blue to green at 5.5 to 6.5). Lysosome morphology and pH will be compared in wild type and chaperone treated and untreated patient cells. This assay can be run in parallel with the plate reader assay to determine the pH-sensitivity. For example, BODIPY-LacCer is trafficked to the Golgi in normal cells, but accumulates in the lysosomes of cells with lipid storage disorders. BODIPY-LacCer fluoresces green or red depending on the concentration in the membrane, and the green/red color ratio in the lysosome can be used to measure changes in concentration. Living healthy cells and patient cells, treated and untreated with compounds, will be incubated with BODIPY-LacCer and the red/green color ratio can be measured by the FACS and/or confocal microscope and the staining pattern (lysosome vs. Golgi) can be determined using a confocal microscope.

Trafficking occurs in cells along pH gradients (i.e. ER pH about 7, Golgi pH about 6.2-7.0, trans-Golgi network pH about 6.0, early and late endosomes pH about 6.5, lysosomes pH about 4.5) and luminal and endosomal pH is disrupted in cells with trafficking defects such as Fabry cells. Accordingly, an assay to determine pH sensitivity in wild type, SPC-treated and untreated patient cells, if correlated to positive effects of pH on trafficking, can be used to monitor restoration of trafficking in Fabry patients. If patient cells are more sensitive to changes in pH, than it would be possible to create a screening assay for SPCs that reduce the cells pH sensitivity, restores lysosome morphology or function, or more generally restores normal trafficking. In addition, mitigation of the trafficking defect can be assessed at the molecular level by determining co-localization of the deficient enzyme (α-Gal A) with a lysosomal marker such as Lyso-Tracker®. Localization of α-Gal A in the lysosome is evidence that trafficking from the ER to the lysosome is restored by treatment with the specific pharmacological chaperone. In brief, normal and patient cells, treated and untreated with SPCs, are fixed and stained with primary antibodies to the enzyme and endosome/lysosome markers (e.g., Rab7, Rab9, LAMP-1, LAMP-2, dystrophin-associated protein PAD) and fluorescently tagged secondary antibodies. The FACS and/or confocal microscope is used to quantify the amount of fluorescence due to the concentration of enzyme and other endocytic pathway markers, and the confocal microscope can be used to determine changes in staining patterns. In addition, traditional biochemical methods, such as pulse-chase metabolic labeling combined with Endoglycosidase H treatment. Endo H only cleaves proteins which have acquired ER glycosylation (high mannose N-linked), i.e., which are localized to the ER, but will not cleave proteins that have made it out of the ER to the Golgi and have acquired additional glycosylation in the Golgi. Accordingly, the greater the level of Endo H sensitive α-Gal A, the more accumulation of the protein in the ER. If the α-Gal A has made it into the Golgi, the glycosidase PNGase F can be used to. confirm whether the protein has exited the Golgi since it cleaves all N-linked sugars.

ER Stress. The toxic accumulation of misfolded proteins in the ER cells, such as the misfolded α-Gal A in Fabry patients, often results in ER stress. This leads to induction of the cell stress response which attempts to resolve the disruption in cell homeostasis. Accordingly, measuring markers of ER stress in patients following treatment with the specific pharmacological chaperone provides another way to monitor the effects of treatment. Such markers include genes and proteins proteins associated with the Unfolded Protein Response, which include BiP, IRE1, PERK/ATF4, ATF6, XBP1 (X-box binding factor 1) and JNK (c-Jun N-terminal kinase). One method to assess ER stress is to compare expression levels between wild type and Fabry patient cells, and also between SPC-treated and untreated cells. ER stress inducers (e.g., tunicamycin for the inhibition of N-glycosylation and accumulation of unfolded proteins in the ER, lacatcystin or H₂O₂) and stress relievers (e.g., cyclohexamide to inhibit protein synthesis) can be used as controls.

Another method contemplated for monitoring the ER stress response is via gene chip analysis. For example, a gene chip with a variety of stress genes can be used to measure expression levels and type of ER stress response (early, late, apoptosis etc.). As one example, the HG-U95A array can be used. (Affymetrix, Inc.).

Lastly, since prolonged ER stress can result in apoptosis and cell death, depending on the level of unfolded proteins in the ER, and the resulting stress level, cells will be more or less sensitive to ER stress inducers such as tunicamycin or proteasome inhibitors. The more sensitive the cells are to the stress inducers, the higher the number of apoptotic or dead cells is observed. Apoptosis can be measured using fluorescent substrates analogs for caspase 3 (an early indicator of apoptosis). FACS, confocal microscopy, and/or using a fluorescence plate reader (96 well format for high through put assays) to determine the percentage of cells positive for apoptosis or cell death (FACS and/or confocal microscopy), or fluorescence intensity can be measured relative to protein concentration in a 96 well format with a fluorescence plate reader.

Another response to cell stress resulting from toxic protein accumulation in the ER is suppression of the ubiquitin/proteasome pathway. This leads to a general disruption of the endocytic pathway (Rocca et al., Molecular Biology of the Cell. 2001; 12: 1293-1301). Misfolded protein accumulation is sometimes correlated with increased amounts of polyubiqutin (Lowe et al., Neuropathol Appl Neurobiol. 1990; 16: 281-91).

Proteosome function and ubiquitination can be assessed using routine assays. For example, evaluation of 26S proteasome function in living animals by imaging has been achieved ubiquitin-luciferase reporter for bioluminescence imaging (Luker et al., Nature Medicine. 2003. 9, 969-973). Kits for proteasome isolation are commercially available from, for example, Calbiochem (Cat. No. 539176). Ubiquitination can be examined by morphological studies using immunohistochemistry or immunofluorescence. For example, healthy cells and patient cells, treated and untreated with SPCs, can be fixed and stained with primary antibodies to ubiquitinated proteins and fluorescence detection of secondary antibodies by FACS and/or confocal microscopy will be used to determine changes in ubiquitinated protein levels.

Another assay to detect ubiquitinated proteins is AlphaScreen™ (Perkin-Elmer). In this model, the GST moiety of a GST-UbcH5a fusion protein is ubiquitinated using biotin-Ubiquitin (bio-Ub). Following ubiquitin activation by E1, in the presence of ATP, bio-Ub is transferred to UbcH5a. In this reaction, UbcH5a acts as the carrier to transfer the bio-Ub to its tagged GST moiety. The protein which becomes biotinylated and ubiquitinated is then captured by anti-GST Acceptor and streptavidin. Donor beads resulting in signal generation. No signal will be generated in the absence of ubiquitination.

Lastly, an ELISA sandwich assay can be used to capture ubiquitinated mutant α-Gal A. The primary antibody to the α-Gal A (e.g., rabbit) would be absorbed to the surface, enzyme would be captured during an incubation with cell lysate or serum, then an antibody (e.g., mouse or rat) to ubiquitinated protein, with secondary enzyme-linked detection, would be used to detect and quantify the amount of ubiquitinated enzyme. Alternatively, the assay could be used to quantify the total amount of multi-ubiquitinated proteins in cell extract or serum.

### Female Carriers

Some female carriers remain asymptomatic and have normal concentrations of α-Gal A, where some experience mild to severe clinical symptoms of disease, especially with increasing age. This variability is thought to be partly the result of inactivation of one X chromosome in some or all cells of the female embryo. In one study of 11 female carriers, proteinuria, acroparaesthesia and angiokeratoma were found to be the most common symptom of the disease (Guffon, Journal of Medical Genetics. 2003; 40: e38). Some of the patients experienced symptoms including vertigo, abdominal pain, depression, exercise/heat intolerance, chilliness or fever and sweating, weakness, and depression. Cardiovascular abnormalities, including valvulopathy and severe hypertrophic myocardiopathy occurred in the fourth, fifth, or sixth decades, and two of the patients experienced terminal renal insufficiency requiring transplantation. One woman had a capsulothalamic stroke, and pulmonary complications (chronic obstructive bronchopneumopathy in one and pulmonary embolism) were also recorded in two patients.

In view of the foreging, the methods of the present invention also can be employed where treatment of female carriers is initiated.

### Combination Therapy

The therapeutic monitoring of the present invention is also applicable following treatment of patients with a combination of DGJ and ERT or gene therapy. Such combination therapy is described in commonly-owned, U.S. patent application publication numbers 2004/0180419 (Ser. No. 10/771,236, and 2004/0219132 (Ser. No. 10/781,356). Both applications are herein incorporated by reference in their entirety.

### Examples

The present invention is further described by means of the examples, presented below. The use of such examples is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification. The invention is therefore to be limited only by the terms of the appended claims along with the full scope of equivalents to which the claims are entitled.

### Example 1

### Administration of DGJ to a Transgenic Mouse Expressing Human Mutant α-Gal A in an Endogenous Enzyme Deficient Background

Transgenic mice that exclusively express human mutant α-Gal A (R301Q) in an α-Gal A knock-out background (TgM/KO mice) were established and evaluated. This Example serves as a biochemical model to study and evaluate pharmacological chaperone therapy for Fabry disease, which is specific for those missense mutations that cause misfolding of α-Gal A.

### Methods

Mice. Fabry R301Q Tg/KO mice were a gift from Dr. Robert Desnick. Male C57BL/6 mice were purchased from Taconic Farms, Germantown, N.Y. and housed in wire cages at 4 mice per cage. All studies were conducted at 8 weeks of age and conducted under strict adherence to IACUC guidelines.

Drug Administration. For the α-Gal A assay, four groups of 10 male C57BL/6 mice were dosed with 0, 1, 10 or 100 mg/kg/day IFG HCl in drinking water for 28 days. For the GL-3 assay, two groups of 6-7 male R301Q Tg/KO mice were dosed daily with 0 or 30 mg/kg DGJ HCl per os for 28 days. After dosing, indicated tissues were harvested and treated as described below.

Enzyme activity assay. After dosing, indicated tissues were harvested and frozen. Tissue lysates were prepared by homogenizing about 50 mg tissue in assay buffer. 2.5 μL lysate was combined with 17.5 μL of the assay buffer and 50 μL of the respective substrate solution (same as detailed in sections above). Reaction mixtures were then incubated at 37° C. for 1 hr. Afterward, 70 μL stop solution (0.4 M glycine, pH 10.8) was added, and fluorescence was read on a Victor plate reader at 355 nm excitation and 460 nm emission. Enzyme activity in the lysates was background subtracted, and normalized for protein concentration using the MicroBCA Protein Assay Kit (Pierce). A 4-MU standard curve was run (same as detailed in sections above) for conversion of fluorescence data to absolute enzyme activity expressed as nmoles/mg protein/hr or further normalized to % of untreated activity.

GL-3 analysis from mouse tissues. Tissue samples were washed free of blood, weighed and homogenized with a solvent system in a FastPrep® system. Homogenate was then extracted using Solid Phase Extraction on a C18 cartridge. The eluent was evaporated and reconstituted prior to injection onto a LC/MS system. Nine GL3 isoforms were measured using positive ESI-MS/MS. LC separation was achieved on a Zorbax C18 column.

### Results

Enzyme Activity. Daily oral gavage administration of DGJ HCl (30 mg/kg; 4 weeks) to male R301Q Tg/KO mice increases mutant GLA activity significantly (*p<0.05, t-test, vs. untreated) in heart, kidney, skin, liver, and spleen tissues (11±0.1, 5.5±0.3, 6.2±0.7, 3±0.02, and 5±0.1 -fold respectively; n=6-7 mice for each tissue). The data is representative of two separate measurements. Two similar experiments were carried out in male and female R301Q Tg/KO mice orally administered DGJ HCl (30 mg/kg/day in drinking water; 4 weeks) and comparable increases in mutant GLA activity were observed in all treated tissues.

Tissue GL-3. Daily oral gavage administration of DGJ HCl (30 mg/kg; 4 weeks) to male R301Q Tg/KO mice reduces GL-3 substrate levels (measured by LC-MS/MS) significantly in skin and heart. DGJ HCl significantly reduced GL-3 levels 2.2-fold after daily per os dosing in skin tissue of treated R301 Q Tg/KO mice (*p<0.05; n=6-7 mice per group). DGJ HCl significantly reduced GL-3 levels 1.6-fold after daily per os dosing in heart tissue of treated R301Q Tg/KO mice (*p<0.01; n=6-7 mice per group). GL-3 levels were also analyzed in kidney tissue and showed a trend towards reduction, but did not reach statistical significance.

### Discussion

The foregoing findings are significant since they represent the first evidence of substrate clearance in vivo in key tissues affected by Fabry disease in response to treatment with a pharmacological chaperone.

As indicated infra, the only approved treatment for Fabry disease is enzyme replacement therapy. Recently, it has been demonstrated that GL-3 persists in heart, kidney, brain (parahippocampus), intestines, adrenal gland, aorta, skin, liver, and spleen from a deceased Fabry patient who had been on long-term ERT therapy with Fabrazyme® (7 years) (Askari et al., manuscript submitted). While this patient did not have GL-3 lysosomal inclusions in vascular endothelial cells, GL-3 immunoreactivity was observed in cell membranes and cytoplasm of cells from all organs. In vascular endothelial cells and fibroblasts of the kidney, GL-3 co-localized with lysosomal, ER, and nuclear markers, and presence in these compartments as well as the cell membrane was confirmed with immunogold electron microscopy. Cultured skin fibroblasts from another Fabry patient on ERT for 7 years showed similar findings. Tissues from three unaffected controls was uniformly negative for GL-3 by IHC and EM. These data suggest that pharmacological chaperone therapy will be as good or better at clearing substrate in key organs in patients with Fabry disease.

In conclusion, a substantial amount GL-3 immunoreactivity remains in cells and tissues even after years of ERT in Fabry disease. For the first time we demonstrate the presence of accumulated globotriaosylceramide in extralysosomal cellular regions. These findings are crucial for the understanding of disease mechanism and suggest the use of immunostaining for globotriaosylceramide to assess response to novel specific therapies.

### Example 2

### Administration of Single Dose DGJ to Evaluate Safety, Tolerability and Pharmacokinetics

This example describes a randomized, double blind, placebo controlled Phase I study of ascending single oral dose of DGJ to evaluate the safety, tolerability and pharmacokinetics of DGJ in healthy volunteers.

Study Design and Duration. This study was first-in-man, single-center, Phase I, randomized, double-blind, single-dose, placebo controlled, ascending dose study to evaluate the safety, tolerability and pharmacokinetics of DGJ following oral administration. The study tested 4 groups of 8 subjects (6 active and 2 placebo) who received a single dose of 25, 75, 225 and 675 mg of DGJ or placebo administered orally, in a dose-escalating regimen, with a minimal 1-week safety evaluation period between successive cohorts. Dose escalation to the next dose level (i.e., next group) proceeded following review of safety and tolerability of the previous group(s). Subjects were housed in the treatment facility from 14 hours prior to dosing until 24 hours after dosing. Meals were controlled by schedule and subjects remained abulatory for 4 hours post drug administration.

Study Population. Subjects were healthy, non-institutionalized, non-smoking male volunteers between 19 and 50 years of age (inclusive) consisting of members of the community at large.

Safety and Tolerability Assessments. Safety was determined by evaluating vital signs, laboratory parameters (serum chemistry, hematology, and urinalysis), ECGs, physical examination and by recording adverse events during the Treatment Period.

Pharmacokinetic Sampling. Blood samples (10 mL each) were collected in blood collection tubes containing EDTA before dosing and at the following times thereafter: 0.25, 0.5, 0.75, 1, 1.5, 2, 3, 4, 5, 6, 8, 10, 12, 16 and 24 hours. Blood samples were cooled in an ice bath and centrifuged under refrigeration as soon as possible. Plasma samples were divided into two aliquots an dstored at 20±10° C. pending assay. At the end of the study, all samples were transferred to MDS Pharma Services Analytical Laboratories (Lincoln) for analysis. The complete urine output was collected from each subject for analysis of DGJ for the following intervals:
−4-0 hours
0-4 hours
4-8 hours
8-12 hours
12-24 hours

Statistical Analysis. Safety data including laboratory evaluations, physical exams, adverse events, ECG monitoring and vital signs assessments were summarized by treatment group and point of time of collection. Descriptive statistics (arithmetic mean, standard deviation, median, minimum and maximum) were calculated for quantitative safety data as well as for the difference to baseline. Frequency counts were compiled for classification of qualitative safety data. In addition, a shift table describing out of normal range shifts was provided for clinical laboratory results. A normal-abnormal shift table was also be presented for physical exam results and ECGs.

Adverse events were coded using the MedDRA version 7.0 dictionary and summarized by treatment for the number of subjects reporting the adverse event and the number of adverse events reported. A by-subject adverse event data listing including verbatim term, coded term, treatment group, severity, and relationship to treatment was provided. Concomitant medications and medical history were listed by treatment.

Pharmacokinetic parameters were summarised by treatment group using descriptive statistics (arithmetic means, standard deviations, coefficients of variation, sample size, minimum, maximum and median). In addition, geometric means were calculated for AUC0-t, AUCinf and Cmax.

### Results

Five subjects (16%) reported 10 treatment-emergent AEs during this study. Two subjects had AEs in Cohort B (75 mg), and three subjects had AEs in Cohort D (675 mg). No subjects presented with AEs in Cohorts A (25 mg) or C (225 mg). No trends were observed with respect to increasing dose levels. No AE was reported by at least 10% of subjects.

AEs occurred in five body systems: the gastrointestinal system (stools watery), musculoskeletal and connective tissues (musculoskeletal stiffness), nervous system (dizziness, headache), respiratory, thoracic and mediastinal system (pharyngolaryngeal pain), and skin and subcutaneous system (rash).

Laboratory deviations from normal ranges occurred after dosing, but none was judged clinically significant. There were no clinically relevant mean data shifts in any parameter investigated throughout the course of the study. No clinically relevant abnormality occurred in any vital sign, ECG, or physical examination parameter.

DGJ appeared to be safe and well tolerated by this group of healthy male subjects as doses increased from 25 mg through 675 mg inclusive.

Pharmacokinetic Evaluation. The following table summarizes the pharmacokinetic data obtained during study.

TABLE 1

| | AUC₀₋ₜ^{a} | AUC_{inf}^{a} | Cₘₐₓ^{a} | tₘₐₓ | t_{1/2} | CL_{R} | Ae^{b} |
|---|---|---|---|---|---|---|---|
| Dose (mg) | (ng * h/mL) | (ng * h/mL) | (ng/mL) | (hours) | (hours) | (L/h) | (%) |
| 25 | 1092 | 1129 | 201 | 3.00 | 3.04 | 5.90 | 8.78 |
| | (34.2) | (33.6) | (35.5) | (21.1) | (15.7) | (1.5) | (173.8) |
| 75 | 4661 | 4730 | 685 | 2.92 | 4.05 | 5.73 | 23.4 |
| | (9.0) | (8.6) | (16.7) | (41.2) | (16.4) | (29.9) | (85.8) |
| 225 | 11177 | 11353 | 1997 | 3.17 | 4.62 | 7.54 | 40.3 |
| | (59.8) | (59.2) | (56.1) | (23.8) | (15.4) | (17.4) | (37.2) |
| 675 | 35275 | 35675 | 6492 | 2.67 | 4.19 | 7.66 | 39.6 |
| | (22.3) | (22.4) | (24.4) | (30.6) | (7.3) | (20.9) | (14.9) |

^{a}Geometric mean was used to present Cₘₐₓ and AUC_{inf} pharmacokinetic parameters.
^{b}Cumulative percentage of DGJ excreted over the 12-hour postdose period.

The pharmacokinetics of DGJ were well characterized in all subjects and at all dose levels. On average, peak concentrations occurred at approximately 3 hours for all dose levels. The mean half-life across all dose levels ranged from approximately 3.0 to 4.6 hours. AUC and Cₘₐₓ of DGJ seemed to increase in a dose-proportional manner when doses were increased from 25 mg to 225 mg.

Mean renal clearance values ranged from 5.90 to 7.66 L/h as doses increased from 25 mg to 675 mg. The mean cumulative percentage of DGJ excreted in urine generally increased as doses increased from 25 mg to 75 mg and were comparable at dose levels of 225 mg and 675 mg.

### Example 3

### Administration of Single Dose DGJ to Evaluate Safety, Tolerability and Pharmacokinetics, and Affect on α-Galatosidase A Enzymatic Activity

This example describes a randomized, double blind, placebo controlled Phase Ib study of twice daily oral doses of DGJ to evaluate the affects of DGJ on safety, tolerability, pharmacokinetics, and α-Galatosidase A (α-Gal A) enzymantic activity in healthy volunteers.

Study Design and Duration. This study was first-in-man, single-center, Phase Ib, randomized, double-blind, twice daily-dose, placebo controlled study to evaluate the safety, tolerability, pharmacokinetics, and α-Gal A enzymantic activity affects of DGJ in healthy volunteers following oral administration. The study tested two groups of of 8 subjects (6 active and 2 placebo) who received a twice daily-dose of 50 or 150 mg b.i.d. of DGJ or placebo administered orally for seven consecutive days, accompanied by a seven day follow up visit. Subjects were housed in the treatment facility from 14 hours prior to dosing until 24 hours after dosing. Meals were controlled by schedule and subjects remained abulatory for 4 hours post drug administration

Pharmacokinetic parameters were calculated for DGJ in plasma on Day 1 and Day 7. In addition, the cumulative percentage of DGJ excreted (12 hours post dose) in urine was calculated. α-Gal A activity was calculated in white blood cells (WBC) before dosing began, and again at 100 hours, 150 hours, and 336 hours into the trial.

Study Population. Subjects were healthy, non-institutionalized, non-smoking male volunteers between 19 and 50 years of age (inclusive) consisting of members of the community at large.

Safety and Tolerability Assessments. Safety was determined by evaluating vital signs, laboratory parameters (serum chemistry, hematology, and urinalysis), ECGs, physical examination and by recording adverse events during the Treatment Period.

Pharmacokinetic Sampling. Blood samples (10 mL each) were collected in blood collection tubes containing EDTA before dosing and at the following times thereafter: 0.25, 0.5, 0.75, 1, 1.5, 2, 3, 4, 5, 6, 7, 8, 9, 10, 11 and 12 hours. Blood samples were cooled in an ice bath and centrifuged under refrigeration as soon as possible. Plasma samples were divided into two aliquots and stored at 20±10° C. pending assay. At the end of the study, all samples were transferred to MDS Pharma Services Analytical Laboratories (Lincoln) for analysis. The complete urine output was collected from each subject for analysis of DGJ to determine renal clearance for the first 12 hours after adiministration of DGJ on days 1 and 7.

WBC α-GAL A Enzymatic Activity Sampling. Blood samples (10 mL each) were collected in blood collection tubes containing EDTA and WBC extracted before dosing and at the following times thereafter: 100 hours, 150 hours, and 336 hours. Samples were treated as described above, and WBC α-Gal A enzymatic activity levels were determined as described in Desnick, R. J. (ed) Enzyme therapy in genetic diseases. Vol 2. Alan R Liss, New York, pp 17-32. Statistical Analysis. Safety data including laboratory evaluations, physical exams, adverse events, ECG monitoring and vital signs assessments were summarized by treatment group and point of time of collection. Descriptive statistics (arithmetic mean, standard deviation, median, minimum and maximum) were calculated for quantitative safety data as well as for the difference to baseline. Frequency counts were compiled for classification of qualitative safety data. In addition, a shift table describing out of normal range shifts was provided for clinical laboratory results. A normal-abnormal shift table was also presented for physical exam results and ECGs.

Adverse events were coded using the MedDRA version 7.0 dictionary and summarized by treatment for the number of subjects reporting the adverse event and the number of adverse events reported. A by-subject adverse event data listing including verbatim term, coded term, treatment group, severity, and relationship to treatment was provided. Concomitant medications and medical history were listed by treatment.

Pharmacokinetic parameters were summarised by treatment group using descriptive statistics (arithmetic means, standard deviations, coefficients of variation, sample size, minimum, maximum and median).

Results. No placebo-treated subjects had AEs and no subject presented with AEs after receiving 50 mg b.i.d. or 150 mg b.i.d. DGJ. DGJ appeared to be safe and well tolerated by this group of healthy male subjects as doses were administered at 50 mg b.i.d. and 150 mg b.i.d.

Laboratory deviations from normal ranges occurred after dosing, but none was judged clinically significant. There were no clinically relevant mean data shifts in any parameter investigated throughout the course of the study. No clinically relevant abnormality occurred in any vital sign, ECG, or physical examination parameter.

Pharmacokinetic Evaluation. The following table summarizes the pharmacokinetic data obtained during study.

TABLE 2

| | 50 mg bid dose | | 150 mg bid dose | |
|---|---|---|---|---|
| | Day 1 | Day 7 | Day 1 | Day 7 |
| Cmax (μM) | 2.3 ± 0.3 | 3.9 ± 0.5 | 11.3 ± 1.5 | 10.8 ± 1.4 |
| tmax (h) | 2.9 ± 0.4 | 2.5 ± 0.4 | 3.1 ± 0.4 | 2.9 ± 0.4 |
| t½ (h) | 2.5 ± 0.1 | | 2.4 ± 0.05 | |
| Cmin (μM) | | 0.4 ± 0.03 | | 1.2 ± 0.1 |
| 12 h cumulative | 16 ± 6 | 48 ± 7 | 42 ± 7 | 60 ± 5 |
| renal excretion | | | | |
| (%)^{a} | | | | |

^{a}Cumulative percentage of DGJ excreted over the 12-hour post dose period.

The pharmacokinetics of DGJ were well characterized in all subjects and at all dose levels. On average, peak concentrations occurred at approximately 3 hours for all dose levels. Cₘₐₓ of DGJ increased in a dose-proportional manner when doses were increased from 50 mg to 150 mg.

The mean elimination half-lives (t₁₁₂) were comparable at dose levels of 50 and 150 mg on Day 1 (2.5 vs. 2.4 hours).

The mean percentage of DGJ excreted over the 12-hour post dose period was 16% and 42% at dose levels of 50 and 150 mg, respectively, on Day 1, increasing to 48% and 60%, respectively, on Day 7.

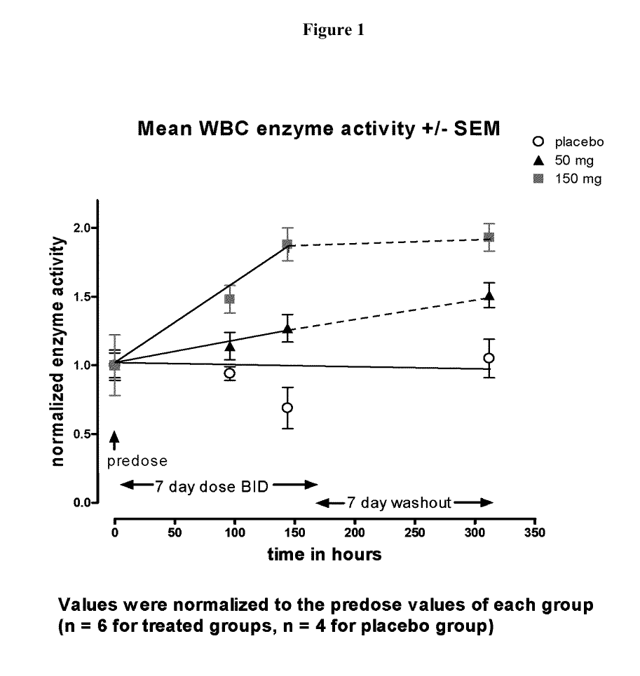

α-Galactosidase A (α-Gal A) Enzymatic Activity. The α-Gal A enzymatic activity data obtained during the study is shown in FIG. 1. DGJ did not inhibit

WBC α-Gal A enzymatic activity in subjects at dosages of 50 mg b.i.d. or 150 mg b.i.d. Furthermore, DGJ produced a dose-dependent trend of increased WBC α-Gal A activity in healthy volunteers. α-Gal A enzymatic levels were measured in WBC of subjects administered placebo, 50 mg b.i.d. DGJ, and 150 mg b.i.d. DGJ. Placebo had no affect on WBC α-Gal A enzymatic levels. Variations in enzymatic levels in response to placebo were not clinically significant. Both 50 mg b.i.d. and 150 mg b.i.d. DGJ increased normalized WBC α-Gal A enzymatic levels. In response to 50 mg b.i.d. DGJ, WBC α-Gal A enzymatic activity increased to 120%, 130%, and 145% pre-dose levels at 100 hours, 150 hours, and 336 hours post-dose, respectively. In response to 150 mg b.i.d. DGJ, WBC α-Gal A enzymatic activity increased to 150%, 185%, and 185% pre-dose levels at 100 hours, 150 hours, and 336 hours post-dose, respectively.

### Example 4

### Administration of DGJ to Fabry Patients and Monitoring the Therapeutic Response

This example describes a Randomized, Blinded Phase II study of DGJ in Fabry patients with α-Gal A folding mutants and proposed monitoring.

Patient enrollment. Fabry patients with known missense mutations in α-Gal A (verified by genotype); patients currently receiving ERT (Fabrazyme®) who are willing to stop ERT for up to 6 months; or newly diagnosed patients who have never been treated with ERT.

Study Design. A two-arm design was planned. In one arm, eligible patients were given 150 mg of DGJ-HCl every other day for 12 weeks, with an extension up to 48 weeks. In the second arm, escalating doses of 25, 100, and then 250 mg b.i.d. over 6 weeks, followed by 50 mg/day for the remainder of the study.

### Monitoring

GL-3 deposits. Skin, kidney and heart biopsies will be performed at baseline, 3 months and six months and evaluated for GL-3 deposits in skin fibroblasts, cardiac myocytes, and various renal cells. It is anticipated that clearance of GL-3 will be observed in all cells. Clearance in cardiac myocytes or renal podocytes or skin tissue has not previously been shown upon treatment with ERT (although changes in urinary sediment at 6 and 18 months of ERT suggested that accumulations of glycosphingolipids in renal tissues were cleared by enzyme replacement; Clin Chim Acta. 2005;360(1-2):103-7).

α-Gal A activity. In addition, α-Gal A activity will be assessed in fractionated tissue obtained from biopsies and in blood leukocytes and plasma (from blood collected at baseline and every month). It is anticipated that DGJ treatment will increase α-Gal A activity from about 2-fold to about 10-fold above baseline in leukocytes, fibroblasts and plasma. It is also anticipated that increases in α-Gal A activity will be observed in tissue, which has not been demostrated with ERT treatment.

Urinalysis. Urine and urinary sediment will be analysed at baseline and monthly for α-Gal A and GL-3. In addition, the abnormal presence of other lipids, such as CTH, lactosylceramide, ceramide, and abnormal decrease or absence of glucosylceramide and sphingomyelin will also be evaluated

Urine will also be analyzed for the presence of protein including albumin (proteinuria) and creatine to monitor the status of renal disease. It is anticipated that DGJ treatment will reduce proteinurea and reduce GL-3 sedimient.

Cardiac anaylsis. In addition to the biopsies described above, MRIs and echocardiogram with strain rate evaluations will be performed at baseline, 3 and 6 months to assess cardiac morphology (e.g., left ventricular hyertrophy) and cardiac function (e.g., congestive heart failure, ischemia, infarction, arrhythmia). Direct reduction in left ventricular hypertrophy, or increase in left ventricular ejection fraction, has never been demonstrated by other treatments. Hypertension will also be ealuated since hypertension (associated with renal dysfunction) can increase the risk for hemorrhagic stroke.

Electrocardiograms will be performed at baseline and at every visit for analysis of improvement in any conduction abnormalities, arrhythmias, bundle branch blocks, or tachy or bradycardia. Previous treatments have not shown improvements in patients presenting with these symptoms.

Renal analysis. Renal podocytes will be evaluated using light and electron microscopy for clearance of GL-3.

Brain analysis. MRI and MRA will be performed at baseline and at the end of the study to assess for a reduction in ischemic arease, which can cause ischemic strokes. The reduction in GL-3 buildup by DGJ is anticipated to reduce the incidence of strokes. Since replacement enzyme cannot cross the blood brain-barrier, improvements in brain ischemia has never been achieved with ERT.

Opthamology. Opthalmologic exams will be performed to assess reduction in corneal and lens opacities such as cataracts.

Neuropathic pain. Subjective patient questionaires will be administered to patients at baseline and at each monthly visit to evaluate reduction in acroparaesthesia. This may be evidece of clearance of GL-3 in the microvasculature of peripheral nerve cells.

Neuropathy. Quantitative Sensory Testing (CASE study) will be used to evaluate peripheral neuropathy.

Hypohidrosis. Sweat glands will be evaluated using quantitative sudomotor axon reflex test (QSART), which assesses the small nerve fiblers that are linked to the eccrine sweat glands. Improvements in the sweat glands should correlate with an increase in sweating, and may also be evidence of clearance of GL-3 in the microvasculature of peripheral nerve cells. This analysis will be performed at baseline and at 3 and 6 months.

### Results

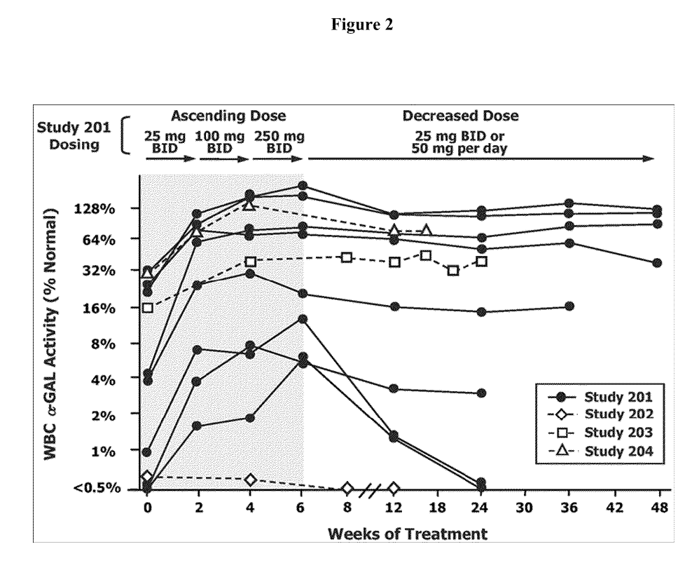

α-Gal A Activity. The available data from the first eleven patients treated with DGJ for at least 12 weeks suggest that treatment with DGJ leads to an increase in the activity of the enzyme deficient in Fabry disease in 10 of the 11 patients (FIG. 2) Three patients in the study received 150 mg of DGJ every other day throughout the entire study (represented on FIG. 2 by open squares, triangles, and diamonds). Eight patients in the study received an acending dose of 25, 100, and then 250 mg b.i.d. over 6 weeks, followed by 50 mg/day for the remainder of the study (represented on FIG. 2 by closed circles). For purposes of calculating the percentage of normal in the table, the level of α-GAL that is normal was derived by using the average of the levels of α-GAL in white blood cells of 15 healthy volunteers from the multiple-dose Phase I study. The 11 patients represented 10 different genetic mutations and had baseline levels of α-Gal A that ranged from zero to 30% of normal.

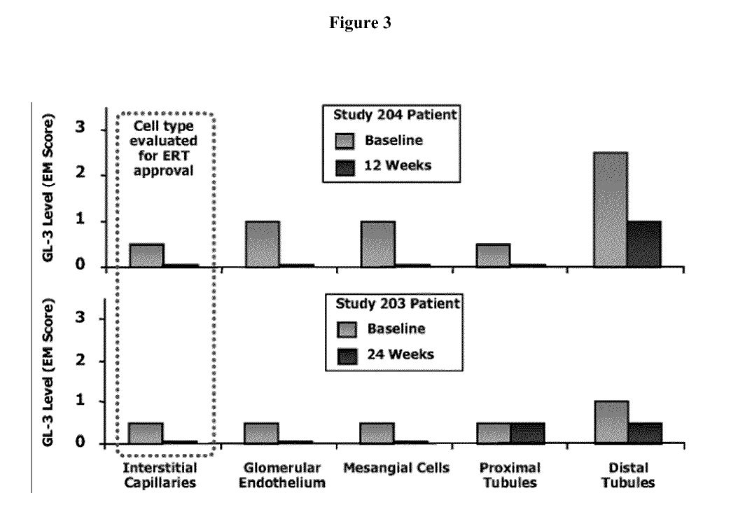

GL-3 levels. Kidney GL-3 levels were assessed by an independent expert using electron microscopy (FIG. 3) Data available for two patients to date showed an observed decrease in GL-3 in multiple cell types of the kidney of one patient after 12 weeks of treatment (mesangial cells and cells of the glomerular endothelium and distal tubules). A second patient showed a decrease of GL-3 levels in the same kidney cell types after 24 weeks of treatment, but these decreases were not independently conclusive because of the patient's lower levels of GL-3 at baseline. Both patients showed a decrease of GL-3 levels in other kidney cell types including cells of the interstitial capillaries, but the decreases were not significant. Urine and plasma GL-3 levels at baseline and after treatment as assessed by liquid chromatography mass spectrometry were available for 10 patients. Most patients had GL-3 levels in urine and plasma that were normal or near normal both before and after treatment. For the few patients that had elevated levels of GL-3 in urine or plasma at baseline, the results were difficult to interpret due to high intra-patient variability.

Skin GL-3 levels at baseline and after treatment as assessed by light and electron microscopy are available for 10 patients. Seven patients had skin GL-3 levels that were normal or near normal both before and after treatment. Results for the three other patients were difficult to interpret because they showed evidence of a decrease in GL-3 in some skin cell types and an increase in GL-3 in other skin cell types, with variability over time.

Most patients in these studies had normal or near normal cardiac, renal and central nervous system function before treatment, and no clinically meaningful changes have been observed after 12 to 48 weeks of treatment.

It is anticipated that more pronounced GL-3 reductions will be evident over longer treatment periods and/or in patients who are more severe and present with higher GL-3 accumulation prior to treatment.

### Discussion

These results represent proof-of-principle for the efficacy of chaperone therapy. DGJ was shown to increase the activity of α-Gal A to levels significantly higher in afflicted individuals that what would have been expected from previous in vitro and transgenic animal studies. Unexpectedly, enzyme activity remains elevated even when the dose was reduced after 6 weeks to the lowest dose (50 mg/day), which demonstrates the potency of the chaperone therapy.

Importantly, since chaperones are small molecules, they easily cross endothelial barriers, such as the blood-brain barrier and those barriers surrounding organs such as the kidney, and they penetrate connective tissue. This represents a significant improvement over ERT, since exogenously adminsitered enzyme does not penetrate these barriers, and therefore has no effect on GL-3 levels in cardiomyocytes, pericytes, and vascular smooth muscle in the heart, renal podocytes, pericytes, perineurium, or the muscular layer of arterioles, histiocytes and fibrocytes (Eng et al., Am J Hum Genet. 2001; 68(3): 711-722). Moreover, as indicated above, it has recently been demonstrated that GL-3 persists in heart, kidney, brain (parahippocampus), intestines, adrenal gland, aorta, skin, liver, and spleen from a deceased Fabry patient who had been on long-term ERT therapy with Fabrazyme® (7 years) (Askari et al., manuscript submitted). Thus,

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, product descriptions, and protocols are cited throughout this application, the disclosures of which are incorporated herein by reference in their entireties for all purposes.

1-46. (canceled)

47. A method for treating a patient with Fabry disease, which comprises determining whether there is an improvement of a surrogate marker that is associated with Fabry disease following administration of a specific pharmacological chaperone of α-galactosidase A.

48. The method of claim 47, wherein the surrogate marker is at least one selected from the group consisting of lysosomal α-galactosidase A activity in cells or tissue; GL-3 levels in kidney, heart or skin tissue; GL-3 levels urine; aberrant trafficking of α-galactosidase A in cells from Fabry patients from the ER to the lysosome; aberrant trafficking of cellular lipids though the endosomal pathway; the presence of increased amounts misfolded α-galactosidase A in the ER or cytosol; the presence of cellular stress resulting from toxic accumulation of α-galactosidase A (as determined by gene and/or protein expression of stress-related markers); aberrant endosomal pH levels; aberrant cell morphology; suppression of the ubiquitin/proteasome pathway; and an increase in the amount of ubiquitinated proteins.

49. The method of claim 48, wherein the surrogate marker is lysosomal α-galactosidase A activity as measured by cytoplasmic staining.

50. The method of claim 49, wherein lysosomal staining is detection of one or both of LAMP-1 expression and polyubiquitinated proteins.

51. The method of claim 47, wherein the pharmacological chaperone is 1-deoxygalactonojirimycin.

52. A method for treating a patient with Fabry disease by adjusting the therapeutic dose of a pharmacological chaperone comprising the steps of measuring the patient's surrogate markers prior to dosing; monitoring the surrogate markers following administration of an initial dose of the pharmacological chaperone of α-galactosidase A, evaluating the effect of the dosage on the surrogate markers and adjusting the dose to obtain surrogate marker levels similar to or trend towards those found in a healthy individual.

53. The method of claim 52, with the further steps of measuring the patient's surrogate markers baseline levels after administering the adjusted dose of the pharmacological chaperone; evaluating the effect of the adjusted dosage on the surrogate markers and further adjusting the dose.

54. The method of claim 52 with the further step of adjusting the dosing regimen.

55. The method of claim 52 wherein the pharmacological chaperone is 1-deoxygalactonojirimycin.

56. The method of claim 47, wherein the patient is a female carrier of Fabry disease.

57. The method of claim 52, wherein the patient is a female carrier of Fabry disease.

58. The method of claim 47 wherein the pharmacological chaperone binds to the alpha-galactosidase A in an amount effective to increase activity of the alpha-galactosidase A in the patient by at least about 2-fold.

59. The method of claim 52 wherein the pharmacological chaperone binds to the alpha-galactosidase A in an amount effective to increase activity of the alpha-galactosidase A in the patient by at least about 2-fold.

60. The method of claim 47 wherein lysosmal levels of globotriaosylceramide are reduced by at least about 2-fold.

61. The method of claim 52 wherein lysosmal levels of globotriaosylceramide are reduced by at least about 2-fold.
